(19) United States
(12) Patent Application Publication
(10) Pub. No.: US 2017/0174649 A1
(43) Pub. Date: Jun. 22, 2017

(54) ESTERS OF ACETALS, COMPOSITION THEREOF, METHODS OF PREPARATION AND MAIN USES THEREOF IN COSMETIC AREA

(71) Applicant: GLYCEROSOLUTION QUIMICA LTDA, Brooklin (BR)

(72) Inventors: Setsuo SATO, São Paulo (SP) (BR); Marcelo Eiji Borges Sato, São Paulo (SP) (BR)

(21) Appl. No.: 15/380,304

(22) Filed: Dec. 15, 2016

(30) Foreign Application Priority Data
Dec. 18, 2015 (BR) 10 2015 031992 4

(51) Int. Cl. C07D 317/24; A61Q 15/00; A61K 8/06; A61K 8/49; A61Q 19/00; A61Q 19/10; A61Q 17/04

(52) U.S. Cl. CPC C07D 317/24; A61Q 19/10; A61Q 15/00; A61Q 17/04; A61K 8/4973; A61Q 19/00; A61K 8/06; A61K 2800/10

(57) ABSTRACT

Esters of acetals of chemical structure:

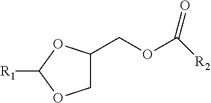

are provided which have excellent solubility power in sunscreens, excellent properties of emollience, spreadability and ease of preparing stable aqueous formulas, are completely non-toxic, have no characteristics that generate ocular or cutaneous irritation, and these properties enable their use as solubilizers and emollients in cosmetic formulations for body use, facial sunscreens, makeup removers and component for use in roll-on deodorants.

### TECHNICAL FIELD

This invention relates to a novel group of monoesters of acetals having, in its structure, the esters of acetals function, produced from glycerin, which give these products excellent properties such as solvency for sunscreens, ease of emulsification, emollience, excellent spreadability without oiliness feature, hydrolytic and chemical stability and low toxicity. These properties make these products excellent candidates as components in cosmetic and personal care formulations like sunscreens, body creams, antiperspirants and deodorants.

### TECHNICAL BACKGROUND

The most commonly used products in the applications mentioned above are those that can solubilize solar ultraviolet radiation protection filters such as dibenzoylmethane derivatives mainly the 4-(1,1-dimethylethyl)-4-metoxydibenzoylmetane (PARSOL 1789) and derivatives of cinnamic acid, in particular 2-ethylhexyl paramethoxycinnamate and octylcinnamate, for the preparation of liquids of stable composition, good spreadability, non-irritability, wash-resistant and especially that generate a comfort sensation in the human skin.

Esters of benzoic acid made with Cl up to C18 carbon chain alcohols, especially those of short chain such as methyl benzoate are quite common; dicapryl ethers, esters of fatty acids such as isopropyl myristate, isopropyl palmitate, octyl octoate. Esters produced from linear or branched carboxylic acids of carbonic chain containing 8 to 18 carbons and acetals produced from glycerin reacted with ketones or aldehydes are the target end products of this invention. Due to the large variety of existing products, the products are selected, in most cases, by the performance, cost and availability of the product in each region. Products from renewable sources may, in some applications, have a higher added value than those from petrochemical source.

Formulations are made using more than one product to achieve the desired effects for each specific need, for example the use of natural antioxidants in cosmetic formulations, which use fatty esters as oil donating agents for the skin. In the industrial area, products and formulations containing effects of lubricity, spreadability, anticorrosive protection, plasticity, adhesiveness, solvency, hydrophilicity, hydrophobicity, are much required by the market.

The cosmetic industry in the production of creams, shampoos and sunscreens uses fatty acid esters such as octyl stearate, isopropyl palmitate, cetyl palmitate, combined with tocopherols to achieve oiliness and protection of the skin and hair.

The domestic and industrial hygiene industry often uses glycols such as butyl glycol or its acetate, monoethylene glycol, monoethylene glycol ether, in cleaning formulations such as detergents, soaps and multi-use cleaners.

In the agricultural industry, it can be verified the use of ethylhexyl lactate, lactamides, toluol, xylol, methyl caprylate, methyl oleate, methyl linoleate, isoparaffins, triacetin, isophorones, citric acid esters, lactic acid esters, Maleic acid, propylene glycol and its diesters, dimethicone, phthalic acid diesters, etc.

Sunscreens and a variety of additives used in cosmetic formulations are solid substances to solubilize. Atoxic products that solubilize and provide good spreadability are the most desired for this type of market. Usually, there are several components looking for the most balanced formula considering the regions, their different ethnicities, climates and economic power so that a greater number of users can benefit from a safe and accessible formula.

U.S. Pat. No. 5,783,173 describes sunscreens and their stable formulations, which use the derivatives of benzoic acid as solubilizer and emollient.

Patent US 20200291010 describes the development of new esters of benzoic acid using branched alcohols whose purpose is to prepare formulations of sunscreens, deodorants and antiperspirants.

U.S. Pat. No. 7,166,739 also describes new esters for cosmetic purposes.

### Objectives of the Invention

The objective of this invention is to create with the glycerin derivatives, motivated by the growing world production thereof, new solubilizing agents of sunscreen since they are the components of greater difficulty of solubilization.

### DETAILED DESCRIPTION OF THE OBJECT

This invention refers to U.S. Patent No. 20140350269 to the same author, describing the synthesis and applications of the esters of acetals, which belongs to the development family previously described in patents PI 0603912-0 and PI 0703673-6 by the same author, where the acetals can be formed in situ, i.e., acetals and esters are formed at the same time and whose glycerin used is the purified glycerin obtained according to the patent BR 1020120015846, although distilled glycerin or ‘blond’ glycerin treated as a market standard can be used in the syntheses of the product contemplated in this invention.

This invention is directed to the use of a specific group of esters of acetals for use in formulations for personal use. It has been shown that esters of acetals containing 12 to 24 total carbons originating from the carbonic chain of the carboxylic acid added to the carbons from the acetals have excellent solubilizing power, are non-irritating to human or ocular skin, are nontoxic, have excellent chemical, hydrolytic stability, good lubricity, low oiliness and generate stable aqueous emulsions and dispersions. These are the initial requirements for candidates for the chemical group called solubilizers and emollients in the cosmetic industry.

These esters are represented by the chemical structure (glycerin acetal esters) below:

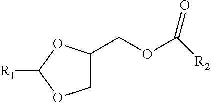

where R1 represents 3 to 8 carbons and R2 represents 8 to 18 carbons.

Table 1 below shows the basic physical and chemical features of cocoil glyceryl acetal used in the solubilizations and formulations tests of the sunscreens.

TABLE 1
(cocoil glyceryl acetal - batch HNC214):

| Aspect | Transparent liquid |
|---|---|
| Active substance | 99.8% |
| Odor | characteristic |
| Freezing point (Celsius) | −3 |
| Flash point (Celsius) | 155 |
| Density 25 Celsius | 0.925 |
| Viscosity@40 Celsius | 8.2 centistokes (cS) |
| Acidity index (mgkoh/g) | 0.2 |
| Saponification index (mgkoh/g) | 178 |
| Gardner color | <1 |

Table 2 below shows solubility of 3 sunscreens obtained in the Market using cocoil glyceryl acetal as a Solubilizing agent.

TABLE 2

| Name of the sunscreen | Solubility at 25° C. grams/100 |
|---|---|
| filter | grams of cocoil glyceryl acetal |
| Ethylhexyl Triazone | 16.0 |
| Diethylamino hydroxybenzoi | 18.0 |
| hexyl benzoate | |
| 4-(1,1-dimethylethyl)-4- | 14.0 |
| methoxydibenzoylmethane | |

In the tests of oral toxicity, the product was classified as class 5 (non-toxic) according to the rules of GHS—“Globally Harmonized Classification for Chemical Substances and Mixtures”.

In the tests of ocular irritation, the product has been classified as non-irritating according to the rules of OECD and GHS.

In the tests of skin irritation, the product has been classified as non-irritating in two tests according to the rules of OECD and GHS, and one of the tests found mild dermal irritation.

Two new batches of cocoil glyceryl acetal were sent to confirm the tests of dermal irritation.

In the following examples, the cocoil glyceryl acetal substance proposed in the invention, was tested in formulations of protective body lotion for ultraviolet radiation (example 1), free-oil face sunscreen (example 2), make up remover (example 3) and formulation of antiperspirant deodorant (example 4), all being compared with products commercially used in the market: C12-C15 Alkyl Benzoate, Dibutyl adipate, Dicaprylyl carbonate.

### Example 1: Body Lotion EPS 30

| Phase | INCI Composition - names | Test A | Test B | Test C | Test D |
|---|---|---|---|---|---|
| I | Xanthan Gum | 0.10 | 0.10 | 0.10 | 0.10 |
| I | Glycerin | 1.00 | 1.00 | 1.00 | 1.00 |
| II | Disodium EDTA | 0.05 | 0.05 | 0.05 | 0.05 |
| II | Acrylates/Beheneth-25 | 0.50 | 0.50 | 0.50 | 0.50 |
| | Methacrylate Copolymer | | | | |
| II | Water | 100 | 100 | 100 | 100 |
| III | Diethylamino | 5.50 | 5.50 | 5.50 | 5.50 |
| | Hydroxybenzoyl | | | | |
| | Hexyl Benzoate | | | | |
| III | Titanium Dioxide (and) | 2.00 | 2.00 | 2.00 | 2.00 |
| | Hydrate Silica (and) | | | | |
| | Dimethicon/Methicone | | | | |
| | Copolymer (and) | | | | |
| | Aluminum Hydroxide. | | | | |
| III | Ethylhexyl | | 9.00 | 9.00 | 9.00 |
| | Methoxycinnamate | | | | |
| III | Cocoil Glyceryl Acetal | 3.00 | — | — | — |
| III | C12-C15 Alkyl Benzoate | — | 3.00 | — | — |
| III | Dibutyl Adipate | — | — | 3.00 | — |
| III | Dicaprylyl Carbonate | — | | | 3.00 |
| III | Caprylic Capric | 3.00 | 3.00 | 3.00 | 3.00 |
| | Triglyceride | | | | |
| III | Cetearyl Alcohol | 2.00 | 2.00 | 2.00 | 2.00 |
| III | Glyceryl Stearate | 2.00 | 2.00 | 2.00 | 2.00 |
| III | Sodium Cetearyl Sulfate | 2.00 | 2.00 | 2.00 | 2.00 |
| III | BHT | 0.10 | 0.10 | 0.10 | 0.10 |
| IV | Aminomethyl Propanol | qs | qs | qs | qs |
| V | Methylene Bis | 0.30 | 0.30 | 0.30 | 0.30 |
| | Benzotriazolyl | | | | |
| | Tetramethylbutylphenol, | | | | |
| | water, Decylglucoside, | | | | |
| | Propylene Glycol, | | | | |
| | xanthan gum. | | | | |
| V | Water | 0.30 | 0.30 | 0.30 | 0.30 |
| VI | Phenoxyethanol (and) | 0.50 | 0.50 | 0.50 | 0.50 |
| | Methylparaben (and) | | | | |
| | Ethylparaben (and) | | | | |
| | Propylparaben (and) | | | | |
| | Butylparaben (and) | | | | |
| | Isobutylparaben | | | | |
| VI | Fragrance | 0.60 | 0.60 | 0.60 | 0.60 |

Process:

1. Weigh the components of phase I, add to Phase II and heat at 80-85° C.

2. Weigh the components of phase III, homogenize and heat at 80-85° C.

3. Pour phase I+II over III under constant stirring.

4. Add Phase IV under stirring up to pH=6.5-7.0, when homogeneous emulsion formation will occur.

5. Separately, premix Phase V and when temperature is below 40° C., add to the emulsion under stirring.

6. Add the components of Phase VI, one by one and homogenize. Adjust the pH (6.5-8.5) if necessary.

Sensory Evaluation—Purpose
Sensory evaluation is a valuable tool for describing creams, lotions, oils, or other compounds from the sensorial point of view.
Several parameters are evaluated by a panel trained under conditions of use.
Along with other physical methods (Corneometer, TEWL) which helps to describe the claims of cosmetic application.

Procedure:
Heated room 21-24° C.
Panel trained with 11 volunteers.

Evaluation is initially effected in 1 min and in 3 minutes after the product application.
It is applied 0.50 g of the test product in the forearm scrubbing with the opposite hand, using the 4 fingers.
It is evaluated the spreadability, absorption, oiliness, softness and smoothness. We use the scales from 1 to 5; wherein 1=Bad and 5=Great.

The formulations B, C and D were subjected to the sensorial panel, compared to formula A, where the results are listed in the Table I below:

| Evaluation | Test A | Test B | Test C | Test D |
|---|---|---|---|---|
| Spreadability | 4 | 2 | 3 | 4 |
| Absorption | 5 | 3 | 3 | 4 |
| Oiliness | 5 | 3 | 4 | 4 |
| Softness | 4 | 4 | 4 | 4 |
| Smoothness | 5 | 3 | 4 | 4 |

The results show that in this formulation the sensorial tests were superior to the market products.

### Example 2: Oil-Free Face Protector

| Phase | Composition -INCI names | Test 1 | Test 2 | Test 3 | Test 4 |
|---|---|---|---|---|---|
| I | Diethylamino | 5.00 | 5.00 | 5.00 | 5.00 |
| | Hydroxybenzoyl Hexyl | | | | |
| | Benzoate | | | | |
| I | Ethylhexyl | 8.50 | 8.50 | 8.50 | 8.5 |
| | Methoxycinnamate | | | | |
| I | Cocoil Glyceryl Acetal | 2.00 | — | — | — |
| I | C12-C15 Alkyl Benzoate | — | 2.00 | — | — |
| I | Dibutyl Adipate | — | — | 2.00 | — |
| | Dicaprylyl Carbonate | — | — | — | 2.00 |
| I | Sodium Acrylates | 3.00 | 3.00 | 3.00 | 3.00 |
| | Copolymer and Paraffinum | | | | |
| | Liquid and PPG-1 | | | | |
| | Trideceth-6 | | | | |
| II | Disodium EDTA | 0.05 | 0.05 | 0.05 | 0.05 |
| II | Water | 100 | 100 | 100 | 100 |
| III | Methylene Bis | 6.0 | 6.00 | 6.00 | 6.00 |
| | Benzotriazolyl | | | | |
| | Tetramethylbutylphenol, | | | | |
| | water, Decylglucoside, | | | | |
| | Propylene Glycol, xanthan | | | | |
| | gum | | | | |
| III | Water | 6.00 | 6.00 | 6.00 | 6.00 |
| IV | Phenoxyethanol (and) | 0.50 | 0.50 | 0.50 | 0.50 |
| | Methylparaben (and) | | | | |
| | Ethylparaben (and) | | | | |
| | Propylparaben (and) | | | | |
| | Butylparaben (and) | | | | |
| | Isobutylparaben | | | | |
| IV | Cyclomethicone | 3.00 | 0.50 | 0.50 | 0.50 |
| IV | Aluminum Starch | 3.00 | 3.00 | 3.00 | 3.00 |
| | Octenylsuccinate | | | | |
| IV | Fragrance | 0.30 | 0.30 | 0.30 | 0.30 |

Procedure:

1. Mix Phase I and Phase II and heat at 80-85° C.

2. Mix them under constant stirring until emulsion formation.

3. Premix Phase III and when temperature is below 40° C., add to the emulsion under constant stirring.

4. Add the components of Phase IV, one by one and homogenize. Adjust the pH (6.5-8.5) if necessary.

The formulations 2, 3 and 4 were subjected to the sensorial panel, compared to the formula 1, where the results are listed in the table below. The protocol used was the same described above, used in the body lotion.

| Evaluation | Test 1 | Test 2 | Test 3 | Test 4 |
|---|---|---|---|---|
| Spreadability | 5 | 3 | 3 | 4 |
| Absorption | 4 | 3 | 4 | 4 |
| Oiliness | 5 | 3 | 4 | 4 |
| Softness | 4 | 4 | 4 | 4 |
| Smoothness | 5 | 3 | 4 | 4 |

### Example 3: Makeup Remover

| Phase | Composition INCI names | Test E | Test F | Test G | Test H |
|---|---|---|---|---|---|
| I | Glyceryl Stearate | 6.00 | 6.00 | 6.00 | 6.00 |
| | (and) Ceteareth- | | | | |
| | 20 (and) Ceteareth12 | | | | |
| | (and) Cetearyl | | | | |
| | Alcohol (and) Cetyl | | | | |
| | Palmitate | | | | |
| I | Cetearyl Alcohol | 0.50 | 0.50 | 0.50 | 0.50 |
| I | Light liquid paraffin | 6.00 | 6.00 | 6.00 | 6.00 |
| I | Isopropyl Palmitate | 6.0 | 6.00 | 6.00 | 6.00 |
| I | Cocoil Glyceryl | 3.00 | — | — | |
| | Acetal | | | | |
| I | C12-C15 Alkyl | — | 3.00 | — | |
| | Benzoate | | | | |
| I | Dibutyl Adipate | — | — | 3.00 | — |
| I | Dicaprylyl Carbonate | — | — | — | 3.00 |
| II | Preservative | 0.30 | 0.30 | 0.30 | 0.30 |
| II | Water (qs) | 100 | 100 | 100 | 100 |

Procedure:

1. Mix Phase I and heat at 80-85″C.

2. Mix Phase II and heat at 80-85° C.

3. Under stirring, add Phase II over Phase I.

4. Cool down under stirring at 30° C. and discharge.

The performance test was performed, where the performance of the emollient was evaluated in the effectiveness of the removal power of lipstick and makeup.

The Makeup Remover formulations were evaluated according to the protocol below:

1. Apply the lipstick on the forearm, making a risk in the same.

2. Apply foundation, making a risk in the same.

3. Let it rest for 10 min.

4. Carry out the removal with a non-woven fabric, moistened with 2 g of the makeup remover to be tested.

5. Evaluate the same, mentioning whether the emollient increased or decreased the power of removal. Considering note 1, as being decreased and 2, increased.

6. Evaluate the sensory in the requirements of softness and smoothness. We used scales from 1 to 5; wherein 1=Bad and 5=Great.

| Evaluation | Test E | Test F | Test G | Test H |
|---|---|---|---|---|
| Removal Power of Makeup | 2 | 2 | 2 | 2 |
| Softness | 5 | 4 | 4 | 4 |
| Smoothness | 5 | 3 | 4 | 4 |

### Example 4 Roll On Deodorant

| Phase | Composition INCI names | Test I | Test J | Test K | Test L |
|---|---|---|---|---|---|
| I. | Glyceryl Stearate | 2.1 | 2.1 | 2.1 | 2.1 |
| | (and) Ceteareth- | | | | |
| | 20 (and) Ceteareth12 | | | | |
| | (and) Cetearyl | | | | |
| | Alcohol (and) Cetyl | | | | |
| | Palmite | | | | |
| | Ceteareth-20 | 1.9 | 1.9 | 1.9 | 1.9 |
| | Glycerin Mono- | 2.0 | 2.0 | 2.0 | 2.0 |
| | Distearate | | | | |
| | Behenyl Alcohol | 1.0 | 1.0 | 1.0 | 1.0 |
| | Ethylhexyl Stearate | 1.7 | 1.7 | 1.7 | 1.7 |
| | Cocoil Glyceryl | 1.7 | — | — | — |
| | Acetal | | | | |
| | C12-C15 Alkyl | — | 1.7 | — | — |
| | Benzoate | | | | |
| | Dibutyl Adipate | — | — | 1.7 | — |
| | Dicaprylyl Carbonate | — | — | — | 1.7 |
| II. | Disodium Edta | 0.05 | 0.05 | 0.05 | 0.05 |
| | Distilled Water (qs) | 100.0 | 100.0 | 100.0 | 100.0 |
| III. | Aluminium | 40.0 | 40.0 | 40.0 | 40.0 |
| | Sesquichlorhydrate | | | | |
| IV. | Fragrance | 0.1 | 0.1 | 0.1 | 0.1 |

Process:

1. Weight Phase I, heat it until 80° 0.

2. Weight Phase II, heat it until 80° C., add phase V over phase II.

3. Pour phases II+V over phase I.

4. Cool the emulsion until 45° C., add phase III and then phase IV.

Protocol:
10 volunteers.
Application in the armpit.
It is evaluated the spreadability, absorption, oiliness, softness and smoothness. We use scales from 1 to 5; wherein 1=Bad and 5=Great.

The formulations J, K and L were subjected to the sensorial panel, compared to the formula I, where the results are listed in the table below.

| Evaluation | Test I | Test J | Test K | Test L |
|---|---|---|---|---|
| Spreadability | 5 | 3 | 3 | 4 |
| Absorption | 5 | 3 | 4 | 4 |
| Oiliness | 5 | 3 | 4 | 4 |
| Softness | 5 | 4 | 4 | 4 |
| Smoothness | 5 | 4 | 4 | 4 |

In all these formulations, Cocoil Gyceryl Acetal demonstrated its solubilizing and/or emollient features in these cosmetic applications.

1-6. (canceled)

7. Esters of acetals of chemical structure: 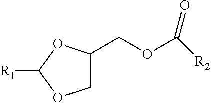
prepared from a substance selected from a group consisting of: oils, coconut fatty acids, babassu, palmiste, and combinations thereof; where:
R1 are the aldehydes and/or ketones containing from 1 to 8 carbons, and
R2 are linear or branched monocarboxylic acids from 2 to 18 carbons,
wherein the carboxylic acids are a mixtures of high purity obtained by a process of enrichment selected from the group consisting of: distillation, extraction and crystallization.

8. The method of claim 7, performed through the esterification or transesterification, wherein acetals are prepared previously or in situ; the reactions of formation of acetals and esters formed concurrently.

9. The method of claim 7, wherein the glycerin acetals are prepared from glycerin of a degrees of purity above 90%.

10. The esters of acetals of claim 7, wherein the substances are glycerin acetal esters.

11. The esters of acetals of claim 10, wherein the substances are monoesters of acetals.

12. The esters of acetals of claim 7, wherein R1 are the aldehydes and/or ketones containing from 3 to 4 carbons.

13. The esters of acetals of claim 7, wherein R2 are linear or branched monocarboxylic acids with at least 12 carbons.

14. The esters of acetals of claim 7, wherein R2 are lauric acids.

15. A personal care composition comprising the glycerin acetal monoesters of claim 7 and an acceptable carrier.

16. A cosmetic formulations for makeup removal comprising the glycerin acetal monoesters of claim 7 and an acceptable carrier.

17. An antiperspirant deodorant comprising the glycerin acetal monoesters of claim 7 and an acceptable carrier.

18. The personal care composition of claim 15, wherein the glycerin acetal monoesters are selected from the group consisting of sunscreen solubilizers against the sun radiation, emollients, spreadability agents in creams, aqueous protective body and face solutions against sun radiation, and oily protective body and face solutions against sun radiation.
